(19) United States
(12) Patent Application Publication
(10) Pub. No.: US 2026/0199048 A1
(43) Pub. Date: Jul. 16, 2026

(54) SHIPPING COVER WITH LOCKING MECHANISM

(71) Applicant: COVIDIEN LP, Mansfield, MA (US)

(72) Inventors: Logamurugaraj SHANMUGAVEL, Pollachi, TN (US); Shaik Ahmed S. HUSSAIN, Hyderabad (IN); Roanit A. FERNANDES, Bethany, CT (US)

(21) Appl. No.: 19/135,003

(22) PCT Filed: Nov. 28, 2023
(86) PCT No.: PCT/IB2023/061964

§ 371 Date: Jun. 2, 2025

Related U.S. Application Data
(60) Provisional application No. 63/431,088, filed on Dec. 8, 2022.

(51) Int. Cl. A61B 90/00; A61B 17/072

(52) U.S. Cl. CPC A61B 90/03; A61B 17/07207; A61B 2017/07271; A61B 2090/038

(57) ABSTRACT

A tool assembly of a surgical stapling device includes a reload assembly and a channel member that defines a cavity to receive the reload assembly. The reload assembly includes a staple cartridge and a shipping cover that is secured to the staple cartridge. The shipping cover includes structure to obstruct removal of the shipping cover from the staple cartridge until after the reload assembly is loaded into the cavity defined by the channel member.

### FIELD

The disclosure is generally related to surgical stapling devices and, more particularly, to surgical stapling devices that include replaceable cartridge assemblies with shipping covers.

### BACKGROUND

Surgical stapling devices for applying rows of staples through compressed living tissue are known in the art and are commonly used for closure of tissue or organs prior to transection or resection of the tissue and for occlusion of organs during thoracic or abdominal procedures. These stapling devices often include a tool assembly that has a cartridge assembly and an anvil that are coupled together and movable in relation to each other between unclamped and clamped positions. The cartridge assembly includes a channel and a staple cartridge that is received within the channel and may be replaceable to facilitate reuse of the stapling device.

Typically, the cartridge assembly includes a channel member and a staple cartridge. The staple cartridge includes a cartridge body defining a plurality of staple pockets and a knife slot, an actuation sled, staple pushers, and staples. The tool assembly includes a knife that may form part of the staple cartridge or the drive assembly. The drive assembly is movable from a retracted position to an advanced position to advance the actuation sled within the cartridge body into engagement with the staple pushers to eject the staples from the staple pockets, and to advance the knife within the cartridge body to cut tissue clamped between the anvil and the cartridge assembly.

Typically, replaceable staple cartridges are stored and shipped separately from the stapling device and are secured within the channel member immediately prior to use of the stapling device. To retain the pushers and staples within the cartridge body of the staple cartridge, it is common to have a shipping cover secured to the cartridge assembly. When the shipping cover is removed from the cartridge assembly prematurely, the staples may be inadvertently discharged from the cartridge body.

A continuing need exists in the art for a shipping cover for a surgical stapling device that prevents premature removal of the shipping cover from the cartridge assembly.

### SUMMARY

This disclosure generally relates to a surgical stapling device including a reload assembly having a staple cartridge and a shipping cover. The shipping cover is secured to the staple cartridge and includes structure to obstruct removal of the shipping cover from the staple cartridge until after the reload assembly is loaded into a channel member of the stapling device.

Aspects of the disclosure are directed to a tool assembly including a reload assembly and a channel member. The reload assembly includes a shipping cover and a staple cartridge. The shipping cover defines a longitudinal axis and includes a cover plate and lock members. The cover plate has outer edges that are substantially parallel to the longitudinal axis. One of the lock members extends downwardly from each of the outer edges of the cover plate. Each of the lock members is formed from a resilient material and includes a locking surface. The lock members are movable from locked positions to unlocked positions. The staple cartridge includes a cartridge body, staples, and pushers. The cartridge body defines a knife slot and staple slots positioned on each side of the knife slot, and the staples and pushers are received within the staple slots. The cartridge body includes a tissue engaging surface, side walls that extend downwardly from opposite sides of the tissue engaging surface, and shoulders that extend along opposite sides of the tissue engaging surface. Each of the side walls of the cartridge body supports a tab that has a substantially planar lower surface. The locking surfaces of the lock members are engageable with the substantially planar lower surface of the tabs to secure the shipping cover to the cartridge body of the staple cartridge. The channel member includes side walls and a bottom wall that define a cavity that receives the staple cartridge. Each of the side walls of the channel member supports a release member and includes an upper surface that supports the shoulders of the cartridge body. The release members are positioned to engage the lock members as the reload assembly is loaded into the cavity of the channel member to move the lock members from the locked positions to the unlocked positions to facilitate separation of the shipping cover from the staple cartridge.

In aspects of the disclosure, the cover plate of the shipping cover includes a lower surface that supports at least one retaining member that is received within the knife slot of the cartridge body to releasably couple the shipping cover to the cartridge body of the staple cartridge.

In some aspects of the disclosure, the at least one retaining member includes raised surfaces that increase frictional contact between the at least one retaining member and the cartridge body.

In certain aspects of the disclosure, the shipping cover includes lock snaps that extend downwardly from each of the outer edges of the cover plate and are configured to engage the shoulders of the cartridge body of the staple cartridge to releasably secure the shipping cover to the cartridge body.

In aspects of the disclosure, each of the lock snaps includes an angled upper surface and the shoulders have an angled lower surface, and the angled upper surface engages the angled lower surface when the shipping cover is coupled to the staple cartridge to releasably secure the shipping cover to the staple cartridge.

In some aspects of the disclosure, each of the side walls of the channel member defines a cutout that is positioned to receive one of the lock members of the shipping cover when the reload assembly is loaded into the cavity of the channel member.

In certain aspects of the disclosure, the release members are positioned within the cutouts.

In aspects of the disclosure, each of the release members includes an abutment that has a radiused engagement surface.

In some aspects of the disclosure, each of the lock members includes a post and a C-shaped catch that has resilient end portions that extend from the post.

In certain aspects of the disclosure, each of the side walls of the cartridge body includes spaced tabs, and the C-shaped catches of the lock members engage the spaced tabs on the side walls of the cartridge body to secure the shipping cover to the cartridge body.

In aspects of the disclosure, the tabs are spaced a distance that is smaller than the width of the C-shaped catches such that the resilient end portions of the C-shaped catches are deformed inwardly and pass by the tabs when the shipping cover is secured to the staple cartridge.

In aspects of the disclosure, each of the side walls of the channel member defines a cutout having a width that is smaller than the width of the C-shaped catch, and the C-shaped catches are received within the cutouts when the reload assembly is loaded into the cavity of the channel member to move the C-shaped catches from locked positions engaged with the tabs to unlocked positions in which the resilient end portions of the C-shaped catches are deformed inwardly of the tabs.

Other aspects of the disclosure are directed to a stapling device including a handle assembly, an elongate body, and a tool assembly. The elongate body has a proximal portion and a distal portion, and the proximal portion is coupled to the handle assembly. The tool assembly includes a reload assembly and a channel member. The reload assembly is supported on the distal portion of the elongate body and includes a shipping cover and a staple cartridge. The shipping cover defines a longitudinal axis and includes a cover plate and lock members. The cover plate has outer edges that are substantially parallel to the longitudinal axis. One of the lock members extends downwardly from each of the outer edges of the cover plate. Each of the lock members is formed from a resilient material and includes a locking surface. The lock members are movable from locked positions to unlocked positions. The staple cartridge includes a cartridge body, staples, and pushers. The cartridge body defines a knife slot and staple slots that are positioned on each side of the knife slot, and the staples and pushers are received within the staple slots. The cartridge body includes a tissue engaging surface, side walls that extend downwardly from opposite sides of the tissue engaging surface, and shoulders that extend along opposite sides of the tissue engaging surface. Each of the side walls of the cartridge body supports a tab that has a substantially planar lower surface. The locking surfaces of the lock members engage the substantially planar lower surface of the tabs to secure the shipping cover to the cartridge body of the staple cartridge. The channel member includes side walls and a bottom wall that define a cavity that receives the staple cartridge. Each of the side walls of the channel member supports a release member and includes an upper surface that supports the shoulders of the cartridge body. The release members are positioned to engage the lock members as the reload assembly is loaded into the cavity of the channel member to move the lock members from the locked positions to the unlocked positions and facilitate separation of the shipping cover from the staple cartridge.

Other aspects of the disclosure are directed to a reload assembly including a shipping cover and a staple cartridge. The shipping cover defines a longitudinal axis and includes a cover plate and lock members. The cover plate has outer edges that are substantially parallel to the longitudinal axis. One of the lock members extends downwardly from each of the outer edges of the cover plate. Each of the lock members is formed from a resilient material and includes a locking surface. The lock members are movable from locked positions to unlocked positions. The staple cartridge includes a cartridge body, staples, and pushers. The cartridge body defines a knife slot and staple slots positioned on each side of the knife slot that receive the staples and pushers. The cartridge body includes a tissue engaging surface, and side walls that extend downwardly from opposite sides of the tissue engaging surface. Each of the side walls of the cartridge body supports a tab that has a substantially planar lower surface. The locking surfaces of the lock members engageable with the substantially planar lower surface of the tabs to secure the shipping cover to the cartridge body of the staple cartridge.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of a surgical stapling device having a tool assembly including a cartridge assembly usable with a shipping cover according to aspects of the disclosure;

FIG. 2 is a side perspective is of the tool assembly shown in FIG. 1 with a staple cartridge of the cartridge assembly of the tool assembly separated from a channel member of the cartridge assembly with a shipping cover secured to the staple cartridge;

FIG. 2A is an enlarged view of the indicated area of detail shown in FIG. 2;

FIG. 3 is a side perspective view of the staple cartridge and shipping cover of the tool assembly shown in FIG. 2 with the shipping cover separated from the staple cartridge;

FIG. 4 is an enlarged view of the indicated area of detail shown in FIG. 3;

FIG. 5 is a bottom perspective view of the shipping cover shown in FIG. 3;

FIG. 6 is an enlarged view of the indicated area of detail shown in FIG. 5;

FIG. 7 is an enlarged view of the indicated area of detail shown in FIG. 2;

FIG. 8 is an enlarged view of the indicated area of detail shown in FIG. 2;

FIG. 9 is a cross-sectional view taken along section line 9-9 of FIG. 5;

FIG. 10 is a cross-sectional view taken along section line 9-9 of FIG. 5;

FIG. 11 is a side perspective view of the tool assembly shown in FIG. 2 as the staple cartridge and the shipping cover are installed into the channel member of the tool assembly;

FIG. 12 is a cross-sectional view taken along section line 12-12 of FIG. 11;

FIG. 13 is a cross-sectional view taken through the cartridge assembly of the tool assembly shown in FIG. 2 with the staple cartridge and the shipping cover installed into the channel member of the tool assembly;

FIG. 14 is a cross-sectional view taken through the cartridge assembly of the tool assembly of the stapling device shown in FIG. 2 with the staple cartridge received in the channel member of the tool assembly as the shipping cover is removed from the staple cartridge;

FIG. 15 is a side perspective view of an alternate version of the staple cartridge and shipping cover of the stapling device shown in FIG. 1 with the shipping cover separated from the staple cartridge;

FIG. 15A is an enlarged view of the indicated area of detail shown in FIG. 15;

FIG. 16 is a side perspective view the staple cartridge and shipping cover shown in FIG. 15 with the shipping cover coupled to the staple cartridge;

FIG. 17 is an enlarged view of the indicated area of detail shown in FIG. 16;

FIG. 18 is a cross-sectional view taken along section line 18-18 of FIG. 17;

FIG. 19 is a side cutaway view illustrating a portion of the shipping cap and staple cartridge and a portion of the channel member as the staple cartridge and shipping cover are loaded into the channel member;

FIG. 20 is a side cutaway view illustrating a portion of the shipping cap and staple cartridge and a portion of the channel member as the staple cartridge and shipping cover are loaded into the channel member;

FIG. 21 is a side perspective view taken through the cartridge assembly shown in FIG. 18 with the staple cartridge received in the channel member of the tool assembly as the shipping cover is removed from the staple cartridge;

FIG. 22 is a perspective view from the proximal end of an alternate version of the staple cartridge and shipping cover according to aspects of the disclosure; and

FIG. 23 is a cross-sectional view taken through the staple cartridge and shipping cover shown in FIG. 22 as the staple cartridge and shipping cover are installed into a channel member of the tool assembly.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. The term “clinician” is used generally to refer to medical personnel including doctors, nurses, and support personnel. In addition, directional terms such as front, rear, upper, lower, top, bottom, and similar terms are used to assist in understanding the description and are not intended to limit the disclosure.

The disclosed surgical stapling device includes a tool assembly that has a reload assembly and a channel member. The reload assembly includes a staple cartridge and a shipping cover that is secured to the staple cartridge. The reload assembly is received within a cavity defined by the channel member. The shipping cover includes structure to obstruct removal of the shipping cover from the staple cartridge until after the reload assembly is loaded into the cavity defined by the channel member.

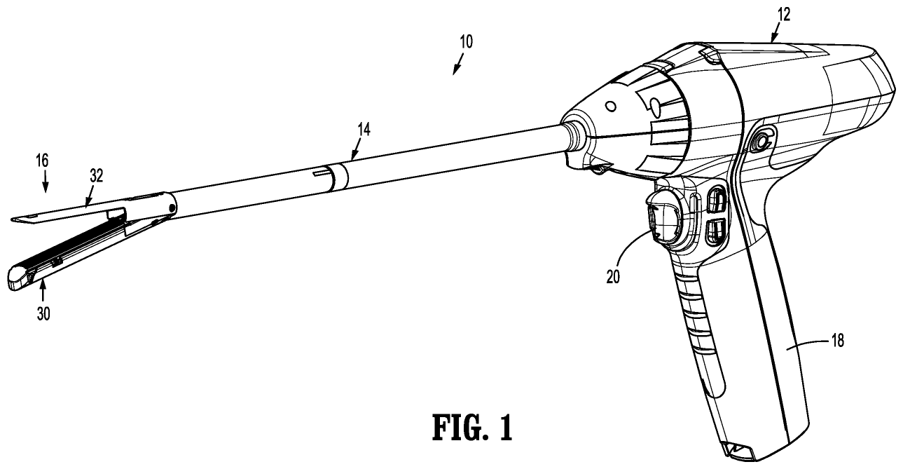

FIG. 1 illustrates a surgical stapling device shown generally as stapling device 10 that includes a handle assembly 12, an elongate body or adapter assembly 14, and a tool assembly 16. As illustrated, the handle assembly 12 is powered and includes a stationary handgrip 18 and an actuation button or actuation buttons, e.g., a rocker 20. The rocker 20 is operable to actuate various functions of the tool assembly 16 via the adapter assembly 14 including approximation, stapling, and cutting. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the features of this disclosure are suitable for use with manually powered surgical stapling devices as well as robotically controlled stapling devices.

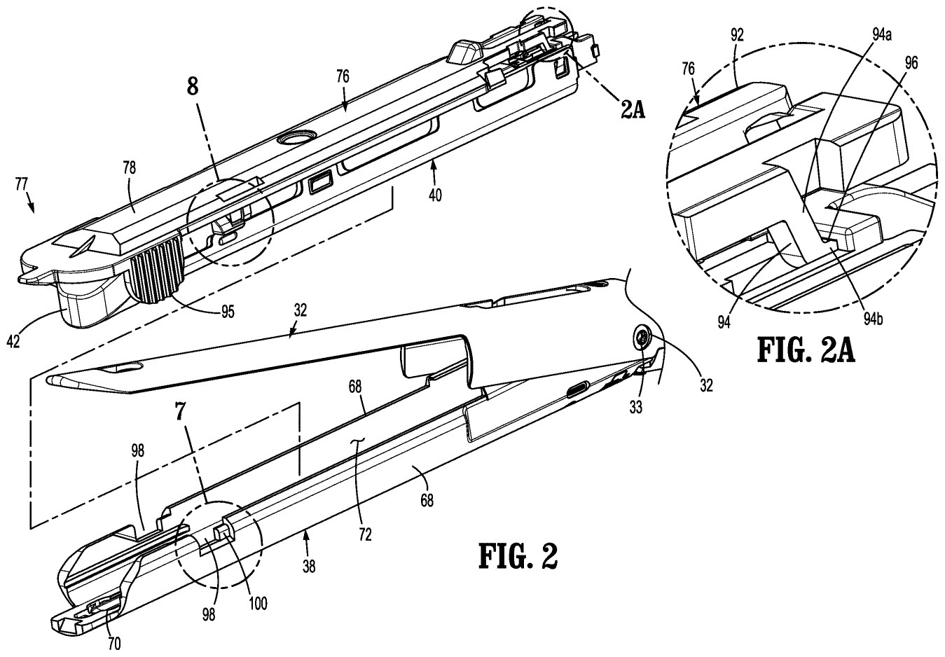

FIG. 2 illustrates the tool assembly 16 of the stapling device 10 which is supported on a distal portion of the adapter assembly 14 and includes a cartridge assembly 30 (FIG. 1) and an anvil 32 that are pivotably coupled together by screws or pins 33. The anvil 32 defines openings 32a that receive the screws or pins 33. The cartridge assembly 30 and the anvil 32 are coupled together such that the cartridge assembly 30 can pivot in relation to the anvil 32 between open and clamped positions. It is envisioned that the anvil 32 and cartridge assembly 30 can be coupled such that the anvil 32 pivots in relation to the cartridge assembly 30.

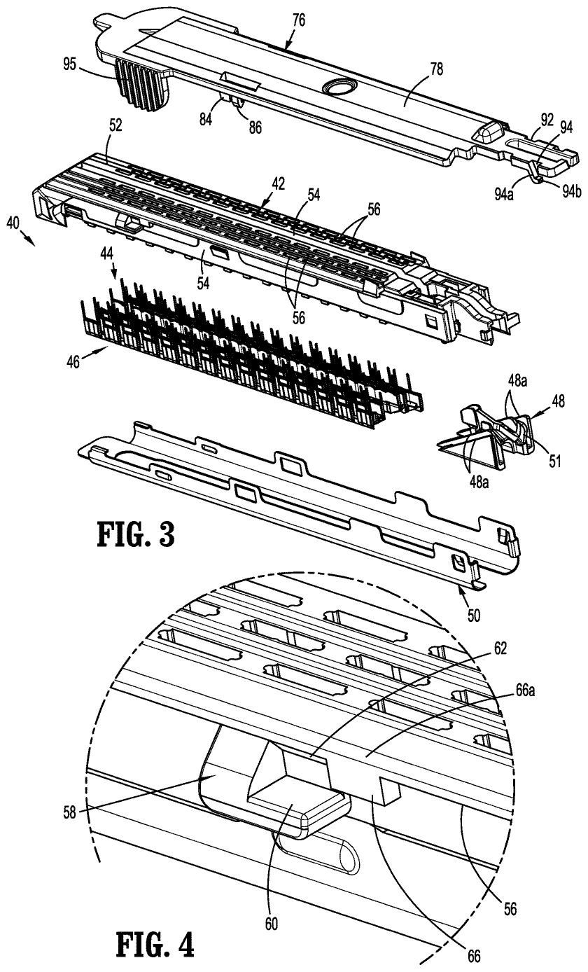

FIGS. 2 and 3 illustrate the cartridge assembly 30 which includes a channel member 38 and a staple cartridge 40. The staple cartridge 40 includes a cartridge body 42, staples 44, pushers 46, an actuation sled 48, and a staple guard 50. The cartridge body 42 includes a tissue engaging surface 52 (FIG. 3) and defines a knife slot 54 and staple slots 56 positioned on opposite sides of the knife slot 54. In certain aspects of the disclosure, the tissue engaging surface 52 can be stepped. The knife slot 54 and the staple slots 56 open onto the tissue engaging surface 52 of the cartridge body 42, and the staples 44 and the pushers 46 are received within the staple slots 56. The actuation sled 48 is received within the cartridge body 42 for movement from a retracted position to an advanced position and includes cam surfaces 48a. The cam surfaces 48a of the actuation sled 48 are positioned to engage the pushers 46 as the actuation sled 48 moves from the retracted position towards the advanced position to eject the staples 44 from the cartridge body 42 into the anvil 32 (FIG. 1).

In aspects of the disclosure, the actuation sled 48 supports a knife 51 (FIG. 3). In some aspects of the disclosure, the knife 51 is pivotably supported on the actuation sled 48 and is movable between lowered and raised positions. The staple guard 50 is secured to a side of the cartridge body 42 opposite to the tissue engaging surface 52 to retain the staples 44 and pushers 46 within the cartridge body 42.

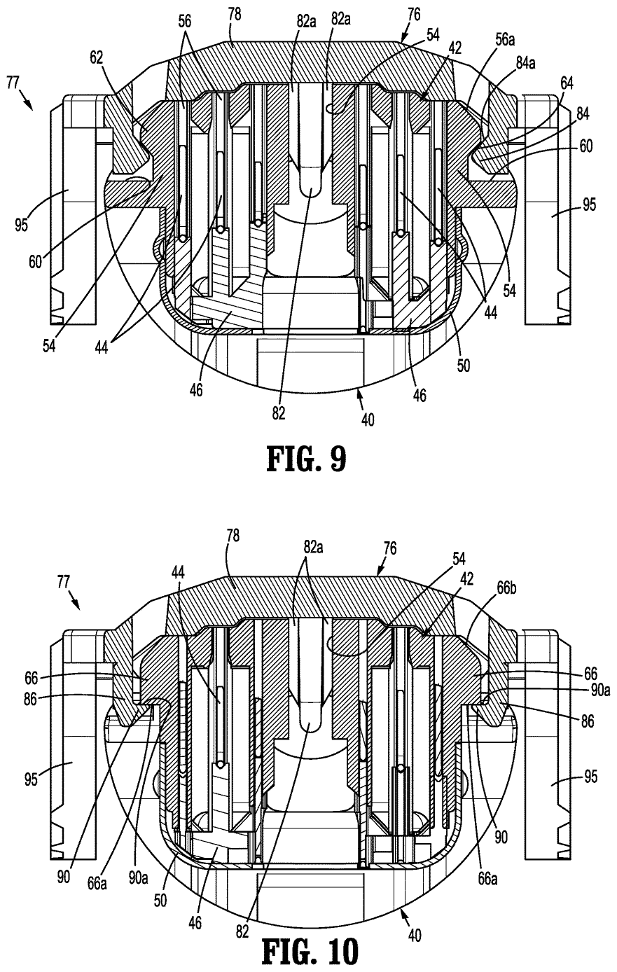

The cartridge body 42 includes side walls 54 that extend downwardly from the tissue engaging surface 52 and shoulders 56 that are positioned between the tissue engaging surface 52 and the side walls 54. The shoulders 56 extend outwardly from the tissue engaging surface 52 and may include a radiused or angled upper surface 56a. The shoulders 56 include lower surfaces are supported on an upper end of side walls 68 (FIG. 2) of the channel member 38 when the staple cartridge 40 is supported within the cavity 72 of the channel member 38. Each of the side walls 54 of the cartridge body 42 includes a protuberance 58 (FIG. 4) and may include a shelf 60 that extends proximally from the protuberance 58. The shoulders 56 of the cartridge body 42 extend outwardly from each of the side walls 54 above the respective shelf 60. The cartridge body 42 also includes a tab 66 (FIG. 10) that is positioned proximally of the shelf 60 and extends downwardly from the shoulder 56 of the cartridge body 42. Each of the shoulders 56 has a lower surface 64 (FIG. 9) that is angled upwardly and outwardly from the side wall 54 of the cartridge body 42 above the shelf 60. In aspects of the disclosure, the tab 66 includes a flat or planar lower surface 66a (FIG. 10).

The channel member 38 (FIG. 2) includes the side walls 68 and a bottom wall 70 that collectively define a cavity 72 that receives the staple cartridge 40. The side walls 68 have a proximal portion that defines openings (not shown) that receive the screws 33 (FIG. 2) to pivotably couple the channel member 38 to the anvil 32. In aspects of the disclosure, the staple cartridge 40 is releasably received within the cavity 72 of the channel member 38 to facilitate replacement of the staple cartridge 40 and reuse of the stapling device 10 (FIG. 1). Alternately, the staple cartridge 40 can be fixedly received within the cavity 46 of the channel member 44. For a more detailed description of the construction and operation of an exemplary cartridge assembly, see U.S. Pat. No. 7,891,534 (the '534 patent).

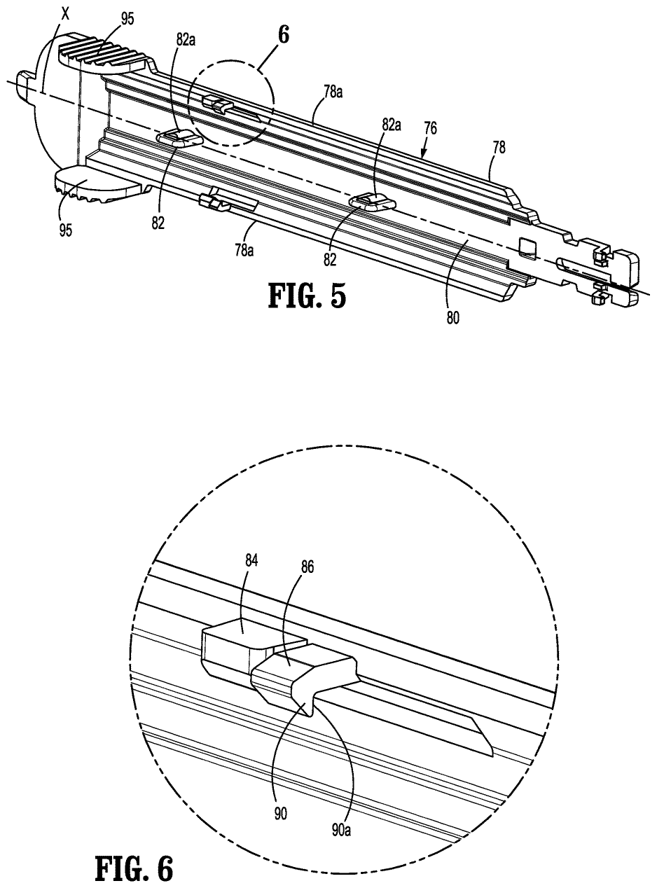

FIGS. 5 and 6 illustrate a shipping cover 76 that is secured to the staple cartridge 40. The staple cartridge 40 and the shipping cover 76 are collectively referred to as the reload assembly 77 (FIG. 2). The shipping cover 76 defines a longitudinal axis “X” (FIG. 5) and includes a cover plate 78 that has a lower surface 80 that is dimensioned to cover the tissue engaging surface 52 of the cartridge body 42. In aspects of the disclosure, the lower surface 80 of the cover plate 78 has a configuration that corresponds to the configuration of the tissue engaging surface 52 of the cartridge body 42. In some aspects of the disclosure, the lower surface of the cover plate 78 and the tissue engaging surface 52 of the cartridge body 42 have stepped configurations. The cover plate 78 includes outer edges 78a that are substantially parallel to the longitudinal axis “X”. The lower surface 80 of the cover plate 78 supports two retaining members 82 that are longitudinally aligned with each other and are received within the knife slot 54 of the cartridge body 42 when the shipping cover 76 is secured to the cartridge body 42. In aspects of the disclosure, the retaining members 82 can include raised surfaces 82a that increase frictional engagement between the retaining members 82 and the cartridge body 42 to secure the shipping cover 76 to the cartridge body 42.

The shipping cover 76 includes hold snaps 84 and lock members or snaps 86. The lock members 86 extend downwardly from outer edges of the cover plate 78 of the shipping cover 76. Each of the hold snaps 84 (FIG. 9) is formed of a resilient material and includes an angled or tapered upper surface 84a (FIG. 9) that is positioned to engage the lower surfaces 64 (FIG. 9) of the shoulders 56 of the cartridge body 42 to releasably secure the shipping cover 76 to the cartridge body 42. The configurations of the surfaces 84a of the hold snaps 84 and the lower surfaces 64 of the shoulders 56 of the cartridge body 42 facilitate release of the hold snaps 84 from the protrusions 62 upon application of a predetermined force to the shipping cover 76.

Each of the lock members 86 includes a locking finger 90 that extends inwardly and includes a locking surface 90a (FIG. 10) that defines a plane that is perpendicular to the side wall 54 of the cartridge body 42 of the staple cartridge 40 and parallel to a plane defined by the planar lower surface 66a of the tab 66 of the cartridge body 42. The lock member 86 is positioned to engage the lower surface 66a of the tab 66 of the cartridge body 42 to lock the shipping cover 76 onto the cartridge body 42.

The shipping cover 76 includes a proximal portion 92 that includes a resilient finger 94. In aspects of the disclosure, the resilient finger 94 includes a first portion 94a that slopes downwardly in a proximal direction from the cover plate 78 and a second portion 94b that extends proximally from the first portion 94a along a plane that is substantially parallel to a plane defined by the cover plate 78. When the shipping cover 76 is secured to the cartridge body 42 of the staple cartridge 40, the second portion 94b of the resilient finger 94 is received in a cutout 96 (FIG. 2A) of the cartridge body 42 to hold the proximal portion of the shipping cover 76 to the proximal portion of the cartridge body 42.

In certain aspects of the disclosure, the shipping cover 76 also includes gripping members 95 that extend downwardly from a distal portion of the cover plate 78 on opposite sides of the cover plate 78. The gripping members 95 can include slip resistant outer surfaces to facilitate grasping by a clinician. In some aspects of the disclosure, the outer surfaces of the grip members 95 are ribbed to facilitate grasping by a clinician. Alternately other slip resistant structure can be included on the grip members 95.

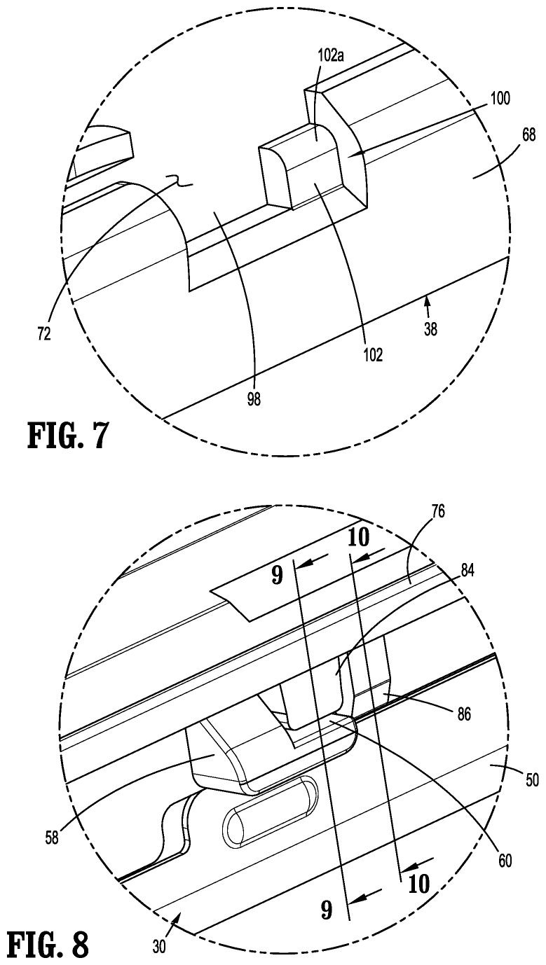

FIGS. 2 and 7 illustrate the channel member 38 which includes the side walls 68. Each of the side walls 68 of the channel member 38 defines a cutout 98. The cutouts 98 are positioned to receive the protuberances 58 and tabs 66 of the cartridge body 42 when the staple cartridge 40 is loaded into the cavity 72 of the channel member 38. The channel member 38 includes release members or structures 100 that are positioned within the cutouts 98. The release structures 100 are positioned to engage the lock members 86 when the reload assembly 77 is loaded into the cavity 72 of the channel member 38 to release the lock members 86 from engagement with the tabs 66 and facilitate removal of the shipping cover 76 from the staple cartridge 40. In aspects of the disclosure, the release structures 100 include abutments 102 that have angled or radiused engagement surfaces 102a.

FIGS. 9 and 10 illustrate the reload assembly 77 with the shipping cover 76 secured to the staple cartridge 40. When the shipping cover 76 is releasably secured onto the cartridge body 42 of the staple cartridge 40, the hold snap 84 flexes outwardly as the hold snap 84 passes over the upper surface 56a of the shoulders 56 of the cartridge body 42 and snaps into position beneath the shoulders 56 to releasably secure the shipping cover 76 to the cartridge body 42 (FIG. 9). When the hold snaps 84 pass over the shoulders 56, the sloped upper surfaces 84a of the hold snaps 84 are engaged with the angled lower surface 64 of the shoulders 56. When the shipping cover 76 is secured to the cartridge body 42 of the staple cartridge 40, the lock members 86 flex outwardly as the lock members 86 passes over a sloped upper surfaces 66b (FIG. 10) of the tabs 66 and snap back to a position in which the locking surfaces 90a of the fingers 90 of the lock members 86 are engaged with the planar lower surfaces 66a of the tabs 66 of the cartridge body 42 to lock the shipping cover 76 onto the staple cartridge 40.

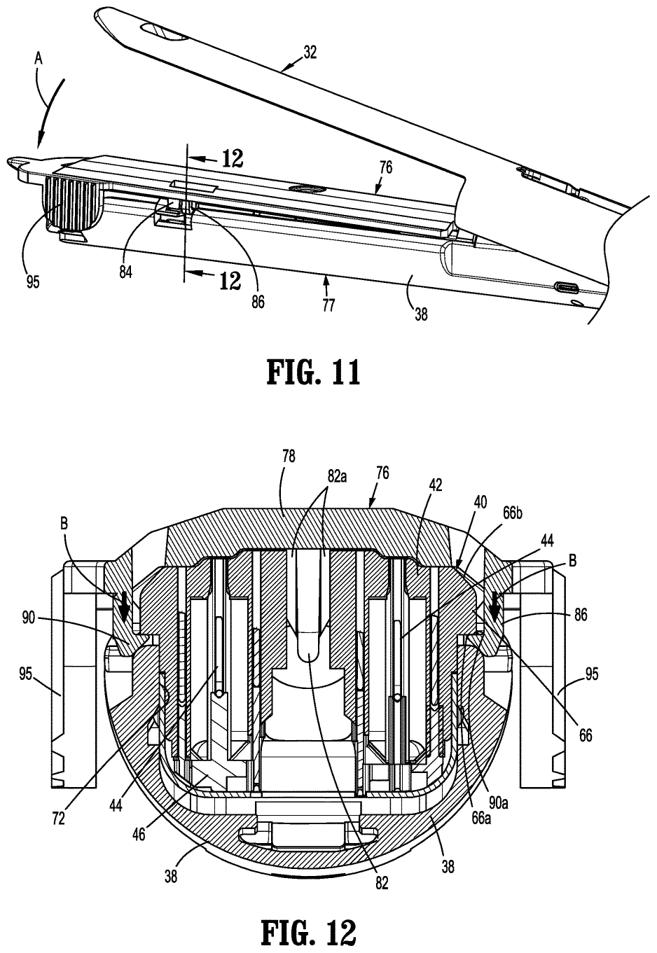

FIGS. 11-13 illustrate the reload assembly 77 as the reload assembly 77 is loaded into the cavity 72 (FIG. 2) of the channel member 38. When the reload assembly 77 is loaded into the cavity 72 of the channel member 38, the proximal portion of the reload assembly 77 is inserted into a proximal portion of the channel member 38 and a distal portion of the reload assembly 77 is rotated downwardly in the direction of arrow “A” in FIG. 11 into the cavity 72 of the channel member 38. As the distal portion of the reload assembly 77 is rotated in the direction of arrow “A” in FIG. 11, the lock members 85 move downwardly in the direction of arrows “B” in FIG. 12 until the locking fingers 90 of the lock members 86 engage the engagement surfaces 102 (FIG. 7) of the release members 100 of the channel member 38. When the locking fingers 90 engage the release members 100 of the channel member 38, the lock members 86 are cammed outwardly in the direction of arrows “C” in FIG. 12 to move the locking fingers 90 outwardly of the planar lower surface 66a of the tab 66 of the cartridge body 42 to unlock the shipping cover 76 from the staple cartridge 40. Once the locking fingers 96 of the lock members 86 are moved to an unlocked position, the gripping members 95 of the shipping cover 76 can be grasped by a clinician and the shipping cover 76 can be pulled upwardly in the direction of arrows “D” in FIG. 14 to separate the shipping cover 76 from the staple cartridge 40. As described above, when a predetermined force is applied to the shipping cover 76 in the direction of arrows “D”, the hold snaps 84 will flex outwardly and pass over the shoulders 56 of the cartridge body 42 of the staple cartridge 40 to allow for separation of the shipping cover 76 from the staple cartridge 40.

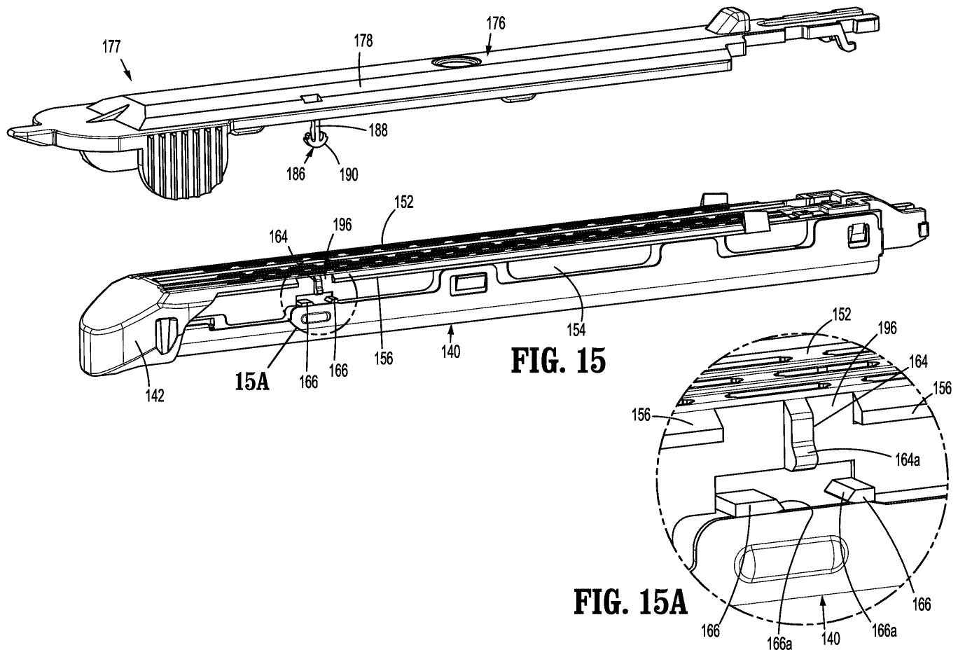

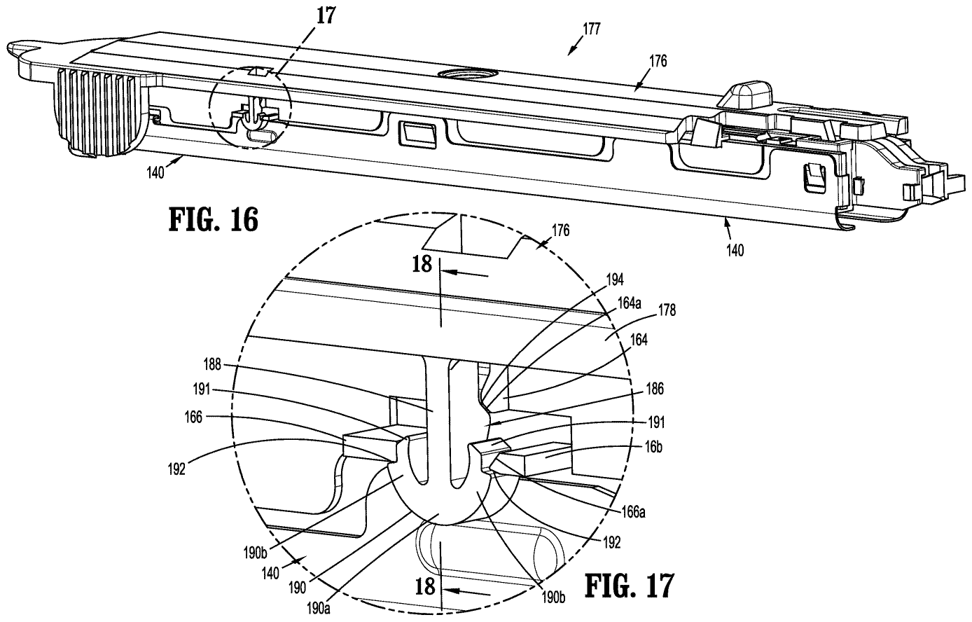

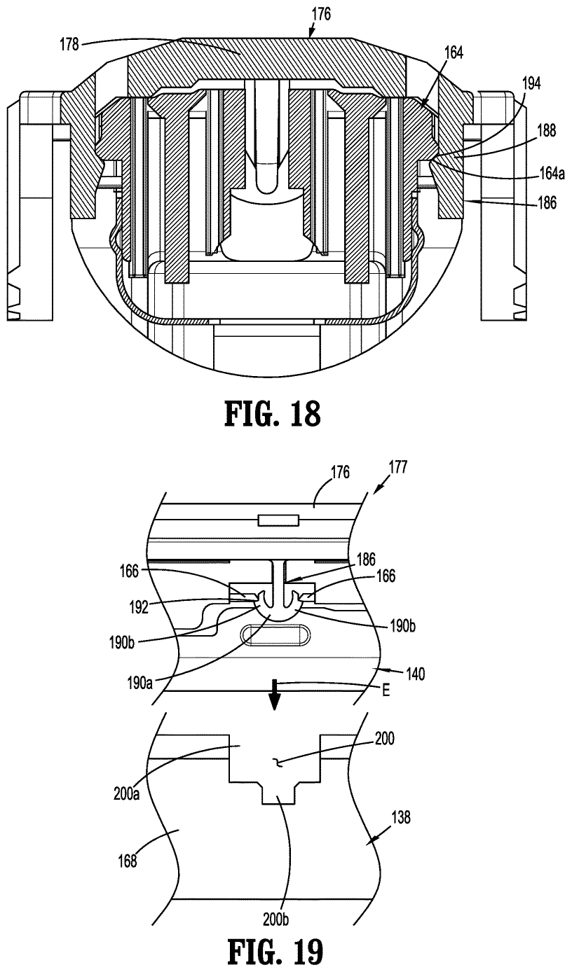

FIGS. 15-21 illustrate an alternate version of the tool assembly 16 (FIG. 1) that includes modifications to the reload assembly 77 (FIG. 2) and to the channel member 38 (FIG. 2) of the cartridge assembly 30. The reload assembly 177 is like the reload assembly 77 and includes a shipping cover 176 and a staple cartridge 140. The shipping cover 176 is as described above regarding the shipping cover 76 except that the configuration of the lock snaps or members 186 are changed. More specifically, the lock members 186 each include a post 188 that extends downwardly from one side of the cover plate 178 and a C-shaped catch 190 that is supported on a lower end of the post 188. The catch 190 includes a central portion 190a (FIG. 17) that is integrally formed with or coupled to the post 188 and opposite end portions 190b that extend or curve upwardly from the central portion 190a towards the cover plate 178. In aspects of the disclosure, each of the end portions 190b of the catch 190 includes a notch 192. The catch 190 is formed from a resilient material to facilitate flexing of the end portions 190b. The post 188 includes an inner surface that includes an angled step 194.

The staple cartridge 140 is like the staple cartridge 40 (FIG. 5) and includes a cartridge body 142 having a tissue engaging surface 152, side walls 154, and shoulders 156. Each of the shoulders 156 defines a slot 196 that is dimensioned to facilitate passage of one of the lock members 186 of the shipping cover 176 as the shipping cover 176 is coupled to the cartridge body 142 of the staple cartridge 140. Each of the side walls 154 of the cartridge body 142 supports a vertical rib 164 and spaced tabs 166 that are positioned below and on opposite sides of the rib 164. The vertical rib 164 includes a lower angled engagement surface 164a (FIG. 15A). The tabs 166 are spaced from each other a distance that is less than the width of the catch 190. When the shipping cover 176 is secured to the staple cartridge 140, the catches 190 of the lock members 186 pass through the spaces defined between the tabs 166 such that the end portions 190b of the catch 190 are deformed inwardly by the tabs 166. When the end portions 190b of the C-shaped catch 190 are inserted through the space between the tabs 166, the end portions 190b snap back to their undeformed position to position the tabs 166 within the notches 192 of the end portions 190b of the catch 190 and lock the shipping cover 176 to the staple cartridge 140. In aspects of the disclosure, end surfaces 166a (FIG. 17) of the tabs 166 are tapered downwardly to allow the catch 190 to pass by the tabs more easily. Similarly, the end surfaces 191 of the end portions 190b of the lock member 186 can also be angled upwardly towards the post 188 of the lock member 186.

When the shipping cover 177 is secured to the staple cartridge 140, the angled step 194 of the post 188 engages the engagement surface 164a of the vertical rib 164 of the cartridge body 142 to releasably secure the shipping cover 176 to the staple cartridge 140. The angled surfaces of the angled step 194 of the post 188 and the angled engagement surface 164a of the vertical rib 164 facilitate release of the shipping cover 177 from the staple cartridge 140 upon application of a predetermined force to the shipping cover 176.

The channel member 138 of the cartridge assembly 130 is similar to the channel member 30 (FIG. 2) and includes side walls 168 (FIG. 19). Each of the side walls 168 defines a stepped cutout 200 that includes a first cutout portion 200a and a second cutout portion 200b. The first cutout portion 200a is dimensioned to facilitate passage of the catch 190. The second cutout portion 200b has a dimension that is smaller than the width of the catch 190 and smaller than the spacing between the tabs 166.

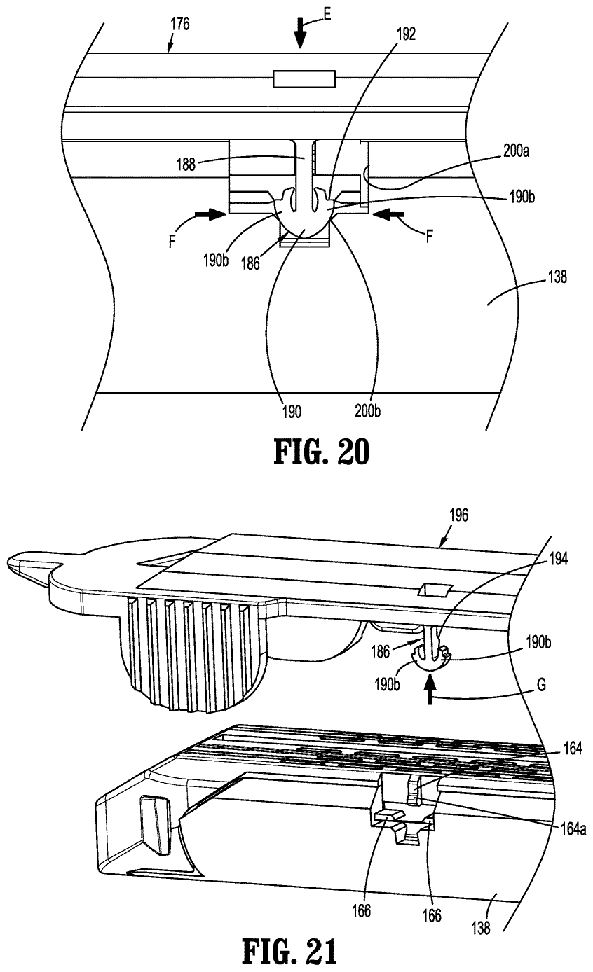

FIGS. 19-21 illustrate the reload assembly 177 as the reload assembly 177 is loaded into the channel member 138. When the reload assembly 177 is loaded into the channel member 138 in the direction of arrow “E” in FIGS. 19 and 20, the C-shaped catches 190 of the lock members 186 initially pass into the first cutout portion 200a of the stepped cutout 200 of the channel member 138 and subsequently move into the second cutout portion 200b. As the reload assembly 177 moves in the direction of arrow “E”, the catches 190 enter the second cutout portion 200b and are deformed inwardly in the direction of arrows “F” in FIG. 20 such that the end portions 190b of the catch 190 can pass between the tabs 166. In this position, upon application of a predetermined force in the direction of arrow “G” in FIG. 21, the shipping cover 176 can be removed from the staple cartridge 140.

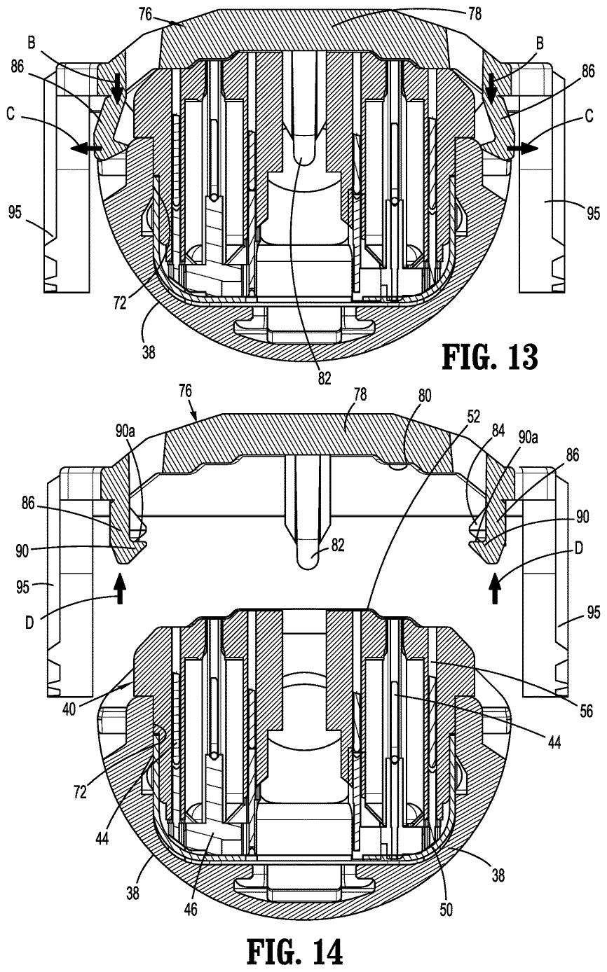

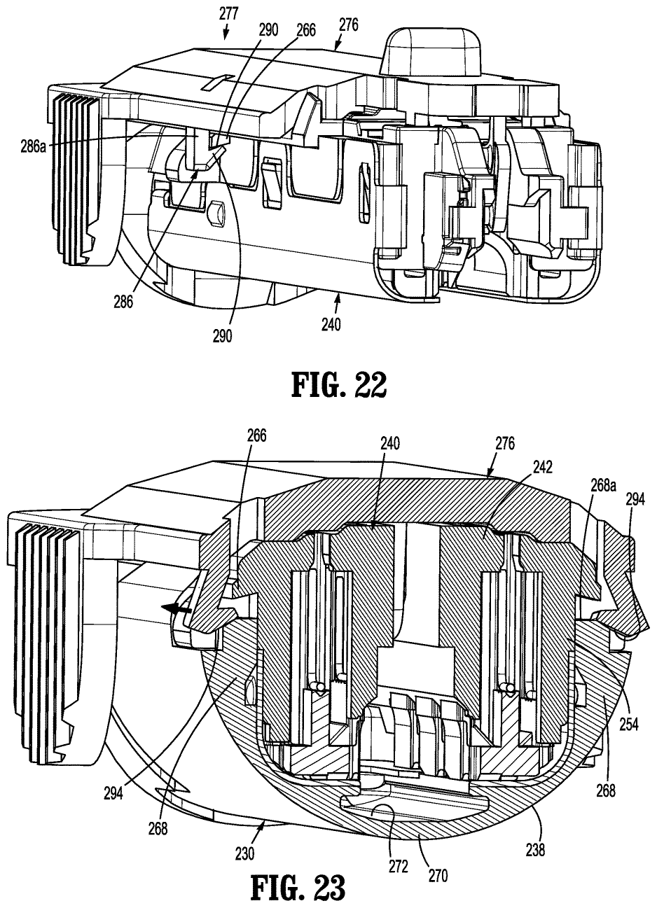

FIGS. 22 and 23 illustrate another alternate version of the tool assembly 16 (FIG. 1) that includes different modifications to the reload assembly 77 (FIG. 2) and to the channel member 38 (FIG. 2) of the cartridge assembly 30. The reload assembly 277 is like the reload assembly 77 and includes a shipping cover 276 and a staple cartridge 240. The shipping cover 276 is as described above regarding the shipping cover 276 except for the configuration of the lock snaps or members 286. More specifically, each of the lock members 286 is like the lock member 86 (FIG. 13) of the shipping cover 276 and includes a body 286a that extends downwardly from an outer edge of the shipping cover 276. Each of the lock members 286 includes a locking finger 290 that extends inwardly from the body 286a and includes a locking surface 290a (FIG. 10) that defines a plane that is substantially perpendicular to the side wall 254 of the cartridge body 242 of the staple cartridge 240 when the shipping cover 276 is secured to the staple cartridge 240. The lock member 286 is positioned to engage a planar lower surface of a tab 266 (FIG. 22) formed on the cartridge body 242 to lock the shipping cover 76 onto the cartridge body 42. Each of the locking fingers 290 of the lock members 286 includes an angled inner surface 294 that defines an acute angle with the planar locking surface 290a of the lock member 286.

The channel member 238 of the cartridge assembly 230 is like the channel member 38 (FIG. 2) and includes side walls 268 and a bottom wall 270 that define a cavity 272. The side walls 268 of the channel member 238 have upper surfaces 268a that include an angled portion 268b. When the reload assembly 277 is loaded into the cavity 272 of the channel member 238, the angled inner surfaces 294 of the locking fingers 290 of the lock members 286 engage the angled portions 268b of the side walls 268 of the channel member 238 to deflect the lock members 286 outwardly in the direction of arrow “H” in FIG. 23 to disengage the lock members 286 from the tabs 266 of the cartridge body 240. Once the lock members 286 are disengaged from the tabs 266, the shipping cover 276 can be lifted from the staple cartridge 240 as described above regarding shipping covers 76 and 176.

Persons skilled in the art will understand that the devices and methods specifically described herein and illustrated in the accompanying drawings are non-limiting exemplary aspects of the disclosed stapling device. It is envisioned that the elements and features illustrated or described in connection with one exemplary aspect of the disclosure may be combined with the elements and features of another without departing from the scope of the disclosure. As well, one skilled in the art will appreciate further features and advantages of the disclosure based on the above-described aspects of the disclosed ligation clip. Accordingly, the disclosure is not to be limited by what has been particularly shown and described, except as indicated by the appended claims.

1. A tool assembly comprising:
a reload assembly including:
a shipping cover defining a longitudinal axis and including a cover plate and lock members, the cover plate having outer edges that are substantially parallel to the longitudinal axis, one of the lock members extending downwardly from each of the outer edges of the cover plate, each of the lock members formed from a resilient material and including a locking surface, the lock members movable from locked positions to unlocked positions; and
a staple cartridge including a cartridge body, staples, and pushers, the cartridge body defining a knife slot and staple slots positioned on each side of the knife slot, the staples and pushers received within the staple slots, the cartridge body including a tissue engaging surface, side walls that extend downwardly from opposite sides of the tissue engaging surface, and shoulders that extend along opposite sides of the tissue engaging surface, each of the side walls of the cartridge body supporting a tab having a substantially planar lower surface, the locking surfaces of the lock members engageable with the substantially planar lower surface of the tabs to secure the shipping cover to the cartridge body of the staple cartridge; and
a channel member including side walls and a bottom wall, the side walls of the channel member and the bottom wall of the channel member defining a cavity that receives the staple cartridge, each of the side walls of the channel member supporting a release member and including an upper surface that supports the shoulders of the cartridge body, the release members positioned to engage the lock members as the reload assembly is loaded into the cavity of the channel member to move the lock members from the locked positions to the unlocked positions to facilitate separation of the shipping cover from the staple cartridge.

2. The tool assembly of claim 1, wherein the cover plate of the shipping cover includes a lower surface that supports at least one retaining member, the at least one retaining member received within the knife slot of the cartridge body to releasably couple the shipping cover to the cartridge body of the staple cartridge.

3. The tool assembly of claim 3, wherein the at least one retaining member includes raised surfaces to increase frictional contact between the at least one retaining member and the cartridge body.

4. The tool assembly of claim 1, wherein the shipping cover includes lock snaps that extend downwardly from each of the outer edges of the cover plate, the lock snaps configured to engage the shoulders of the cartridge body of the staple cartridge to releasably secure the shipping cover to the cartridge body.

5. The tool assembly of claim 4, wherein each of the lock snaps includes an angled upper surface and the shoulders have an angled lower surface, the angled upper surface engaging the angled lower surface when the shipping cover is coupled to the staple cartridge to releasably secure the shipping cover to the staple cartridge.

6. The tool assembly of claim 1, wherein each of the side walls of the channel member defines a cutout that is positioned to receive one of the lock members of the shipping cover when the reload assembly is loaded into the cavity of the channel member, the release members positioned within the cutouts.

7. The tool assembly of claim 6, wherein each of the release members includes an abutment, the abutments having radiused engagement surfaces.

8. The tool assembly of claim 1, wherein each of the lock members includes a post and a C-shaped catch having resilient end portions that extend from the post.

9. The tool assembly of claim 8, wherein each of the side walls of the cartridge body includes spaced tabs, the C-shaped catches of the lock members engaged with the spaced tabs on the side walls of the cartridge body to secure the shipping cover to the cartridge body.

10. The tool assembly of claim 9, wherein the tabs are spaced a distance that is smaller than the width of the C-shaped catches such that the resilient end portions of the C-shaped catches are deformed inwardly and pass by the tabs when the shipping cover is secured to the staple cartridge.

11. The tool assembly of claim 10, wherein each of the side walls of the channel member defines a cutout, the cutouts having a width that is smaller than the width of the C-shaped catches, the C-shaped catches received within the cutouts when the reload assembly is loaded into the cavity of the channel member to move the C-shaped catches from locked positions engaged with the tabs to unlocked positions in which the resilient end portions of the C-shaped catches are deformed inwardly of the tabs.

12. A stapling device comprising:
a handle assembly;
an elongate body having a proximal portion and a distal portion, the proximal portion coupled to the handle assembly; and
a tool assembly including:
a reload assembly supported on the distal portion of the elongate body, the reload assembly including:
a shipping cover defining a longitudinal axis and including a cover plate and lock members, the cover plate having outer edges that are substantially parallel to the longitudinal axis, one of the lock members extending downwardly from each of the outer edges of the cover plate, each of the lock members formed from a resilient material and including a locking surface, the lock members movable from locked positions to unlocked positions; and
a staple cartridge including a cartridge body, staples, and pushers, the cartridge body defining a knife slot and staple slots positioned on each side of the knife slot, the staples and pushers received within the staple slots, the cartridge body including a tissue engaging surface, side walls that extend downwardly from opposite sides of the tissue engaging surface, and shoulders that extend along opposite sides of the tissue engaging surface, each of the side walls of the cartridge body supporting a tab having a substantially planar lower surface, the locking surfaces of the lock members engageable with the substantially planar lower surface of the tabs to secure the shipping cover to the cartridge body of the staple cartridge; and
a channel member including side walls and a bottom wall, the side walls of the channel member and the bottom wall of the channel member defining a cavity, the staple cartridge received within the cavity, each of the side walls of the channel member supporting a release member and including an upper surface that supports the shoulders of the cartridge body, the release members positioned to engage the lock members as the reload assembly is loaded into the cavity of the channel member to move the lock members from the locked positions to the unlocked positions and facilitate separation of the shipping cover from the staple cartridge.

13. The stapling device of claim 12, wherein each of the side walls of the channel member defines a cutout that is positioned to receive one of the lock members of the shipping cover when the reload assembly is loaded into the cavity of the channel member.

14. The stapling device of claim 13, wherein each of the release members is positioned within one of the cutouts of the channel member.

15. The stapling device of claim 14, wherein each of the release members includes an abutment, the abutment having a radiused engagement surface.

16. The stapling device of claim 15, wherein each of the lock members includes a post and a C-shaped catch having resilient end portions that extend from the post.

17. The stapling device of claim 12, wherein each of the side walls of the cartridge body includes spaced tabs, the C-shaped catches of the lock members engaged with the spaced tabs on the side walls of the cartridge body to secure the shipping cover to the cartridge body.

18. The stapling device of claim 17, wherein the tabs are spaced a distance smaller than the width of the C-shaped catch such that the resilient end portions of the C-shaped catch are deformed inwardly and pass by the tabs when the shipping cover is secured to the staple cartridge.

19. The stapling device of claim 18, wherein each of the side walls of the channel member defines a cutout, the cutout having a width that is smaller than the width of the C-shaped catch, the C-shaped catch received within the cutout when the reload assembly is loaded into the cavity of the channel member to move the C-shaped catch from a locked position engaged with the tabs to an unlocked position in which the resilient end portions of the C-shaped catch are deformed inwardly of the tabs.

20. A reload assembly comprising:
a shipping cover defining a longitudinal axis and including a cover plate and lock members, the cover plate having outer edges that are substantially parallel to the longitudinal axis, one of the lock members extending downwardly from each of the outer edges of the cover plate, each of the lock members formed from a resilient material and including a locking surface, the lock members movable from locked positions to unlocked positions;
a staple cartridge including a cartridge body, staples, and pushers, the cartridge body defining a knife slot and staple slots positioned on each side of the knife slot that receive the staples and pushers, the cartridge body including a tissue engaging surface and side walls that extend downwardly from opposite sides of the tissue engaging surface, each of the side walls of the cartridge body supporting a tab that has a substantially planar lower surface, the locking surfaces of the lock members engageable with the substantially planar lower surface of the tabs to secure the shipping cover to the cartridge body of the staple cartridge.
